(19) United States
(12) Patent Application Publication
(10) Pub. No.: US 2008/0125842 A1
(43) Pub. Date: May 29, 2008

(54) THERMAL, ELASTIC, TIGHT-FITTING GARMENT WITH POCKETS POSITIONED FOR THERMAL THERAPY

(76) Inventor: Steve Petitt, Atlantic Beach, FL (US)

Correspondence Address: MARK YOUNG, P.A., 12086 FORT CAROLINE ROAD, UNIT 202, JACKSONVILLE, FL, 32225, US

(21) Appl. No.: 11/744,257

(22) Filed: May 4, 2007

Related U.S. Application Data
(60) Provisional application No. 60/746,487, filed on May 4, 2006.

(51) Int. Cl. A61F 7/00; A41D 1/00; A41D 27/20; B65D 69/00

(52) U.S. Cl. 607/108; 607/114; 2/69; 2/250; 206/570

(57) ABSTRACT

A form-fitting elastic garment includes a plurality of pockets at locations corresponding to a plurality of muscle groups and/or tendons. The pockets are adapted to receive a thermal medium and hold the thermal medium tightly against a muscle group and/or tendons of a wearer. A thermal therapy kit includes a thermally insulated container storing a thermal medium and such a form fitting garment.

### RELATED APPLICATION

This application claims the benefit of priority of U.S. provisional application 60/746,487, filed May 4, 2006, the entire contents of which are incorporated herein by this reference.

### FIELD OF THE INVENTION

This invention relates generally to garments, and, more particularly, to tight fitting garments with pockets configured for storing thermal media such as ice packs tightly against determined anatomical areas to facilitate recovery after strenuous activity.

### BACKGROUND

Thermal therapy conventionally involves the application of heat or cold to tissue in an effort to heal and rehabilitate injuries such as bruises, sprains, or other trauma to bone, muscle, ligaments, tendons, and tissue and to treat degenerative conditions and inflammatory diseases and disorders. In a cold application, a cold medium is applied to an affected area to diminish swelling and inflammation to resultantly reduce pain and promote healing of injured tissue. In a heat application, a hot medium is applied to an affected area to loosen extremity tissue and joint tissue, such as muscles, ligaments and tendons. Application of heat promotes repair and healing by increasing the temperature of the affected area, thereby increasing the flow of blood and oxygen to the tissue and increasing respiration. In this manner, the application of heat serves to increase the range of motion and improve the flexibility in the patient's extremity, thus improving the functionality, comfort and performance of the injured or infirm targeted area.

Athletes are always searching for improved methods to expedite recovery from grueling training and events. A recovery method which has become the choice (and bane) of many athletes is an ice bath, which is a form of thermal therapy. After intense exercise athletes have microscopic tears in their muscles that cause inflammation. The chilling temperature of an ice bath is a vasoconstrictor, causing blood vessels to contract and drain blood from chilled areas. After leaving the bath, fresh blood flows into the previously contracted vessels, invigorating the muscles with oxygen and in turn, reducing inflammation. This increase in blood flow also makes its way deep into the muscle tissue, helping flush out any build-up of lactic acid. The combined effect of supplying oxygen to muscles while flushing out lactic acid facilitates recovery of muscles, tendons, bones, nerves and other tissue.

Unfortunately, there are many disadvantages to ice baths. For example, athletes dread the intense discomfort and severe pain caused by the frigid ice and water. Another disadvantage is that an ice bath indiscriminately chills the entire submerged portions of the body. Therefore, not only are the affected muscle groups exposed to the cold temperatures, but all neighboring parts of the anatomy must endure the intense cold of an ice bath, even if no benefit is attainable for those neighboring parts. Additionally, ice baths are most effective when taken within about 60 minutes of finishing rigorous activity. However, they are not portable and are often inaccessible after an event.

Concomitantly, other conventional thermal therapies do not facilitate targeting a plurality of muscles, ligaments, tendons, and tissues simultaneously with a thermal medium. Instead, such other conventional thermal therapies typically entail wrapping one targeted area at a time. The wraps may be difficult to apply, and frequently shift after they are applied. Consequently, an exhausted athlete using such conventional therapies may be able to target only one of several muscle groups requiring therapy. Only after the window of therapeutic opportunity has passed would the athlete be able to target the other muscle groups.

The invention is directed to fulfilling one or more of the needs and overcoming one or more of the problems as set forth above.

### SUMMARY OF THE INVENTION

To overcome one or more of the problems as set forth above, in one aspect of the invention, a form-fitting pant garment is provided. The garment comprises an elastic material with an outer side. A plurality of pockets are attached to the pant garment on the outer side at locations corresponding to a plurality of muscle groups. The pockets are adapted to receive a thermal medium and hold the thermal medium tightly against a muscle group of a wearer.

In another aspect of the invention, a form-fitting shirt garment is also provided. The garment comprises an elastic material with an outer side. A plurality of pockets are attached to the shirt garment on the outer side at locations corresponding to a plurality of muscle groups. The pockets are adapted to receive a thermal medium and hold the thermal medium tightly against a muscle group of a wearer.

In yet another aspect of the invention, a form-fitting sock garment is also provided. The garment comprises an elastic material with an outer side. A pocket is attached to the sock garment on the outer side at a locations corresponding to a muscle group. The pocket is adapted to receive a thermal medium and hold the thermal medium tightly against a muscle group of a wearer.

In yet another aspect of the invention, a thermal therapy kit is provided. The kit includes a thermally insulated container adapted for storing a thermal medium and a form fitting garment. The kit also includes a form-fitting garment comprising an elastic material with an outer side. A plurality of pockets are attached to the garment on the outer side at locations corresponding to a plurality of muscle groups. The pockets are adapted to receive a thermal medium and hold the thermal medium tightly against a muscle group of the wearer. The kit also includes a plurality of thermal media.

In one embodiment, the form-fitting garment includes an elastic material with an outer side, a plurality of pockets attached to the garment on the outer side at locations corresponding to a plurality of muscle groups, each of the pockets being adapted to receive a thermal medium and hold the thermal medium tightly against a muscle group of the wearer, and each of the pockets further being sized to cover at least a substantial portion of the muscle group.

In another embodiment the garment is a short-sleeve shirt and the plurality of pockets include shoulder pockets, the shoulder pockets being positioned on areas of the shirt corresponding to shoulders of an intended wearer. In another embodiment the short-sleeve shirt includes a lumbar pocket, the lumbar pocket being positioned on an area of the shirt corresponding to a lumbar region of an intended wearer. In another emodiment, the short-sleeve shirt includes a trapezius pocket, the lumbar pocket being positioned on an area of the shirt corresponding to a trapezius region of an intended wearer.

In another embodiment the garment is a long-sleeve shirt and the plurality of pockets include shoulder pockets, the shoulder pockets being positioned on areas of the shirt corresponding to shoulders of an intended wearer. The long-sleeve shirt may also include a lumbar pocket, the lumbar pocket being positioned on an area of the shirt corresponding to a lumbar region of an intended wearer. The long-sleeve shirt may also include a trapezius pocket, the lumbar pocket being positioned on an area of the shirt corresponding to a trapezius region of an intended wearer. The long-sleeve shirt may also include upper arm pockets, the upper arm pockets being positioned on an area of the shirt corresponding to upper arm regions of an intended wearer. The long-sleeve shirt may also include elbow pockets, the elbow pockets being positioned on an area of the shirt corresponding to elbow regions of an intended wearer. The long-sleeve shirt may also include forearm pockets, the forearm pockets being positioned on an area of the shirt corresponding to forearm regions of an intended wearer.

In another embodiment the garment is shorts and the plurality of pockets include quadriceps pockets, the quadriceps pocket being positioned on an area of the shorts corresponding to quadriceps regions of an intended wearer. The plurality of pockets may also include hamstring pockets, the hamstring pocket being positioned on an area of the shorts corresponding to hamstring regions of an intended wearer.

In another embodiment the garment is pants and the plurality of pockets include quadriceps pockets, the quadriceps pocket being positioned on an area of the pants corresponding to quadriceps regions of an intended wearer. The plurality of pockets include hamstring pockets, the hamstring pocket being positioned on an area of the pants corresponding to hamstring regions of an intended wearer.

The plurality of pockets may also include calf pockets, the calf pocket being positioned on an area of the pants corresponding to calf regions of an intended wearer. The plurality of pockets may also include ankle pockets, the ankle pocket being positioned on an area of the pants corresponding to ankle regions of an intended wearer.

In another embodiment the garment is a pair of socks and the plurality of pockets include ankle pockets, the ankle pockets being positioned on areas of the socks corresponding to ankle regions of an intended wearer.

In another embodiment the garment is a sleeve and the plurality of pockets include an upper arm pocket and an elbow pocket the upper arm pocket being positioned on areas of the sleeve corresponding to upper arm regions of an intended wearer, and the elbow pocket being positioned on areas of the sleeve corresponding to elbow regions of an intended wearer.

In another embodiment, a kit is provided. The kit includes a thermally insulated container adapted for storing a thermal medium and a form fitting garment. The kit also includes a form-fitting garment stored in the thermally insulated container. The garment includes an elastic material with an outer side, a plurality of pockets attached to the garment on the outer side at locations corresponding to a plurality of muscle groups, each of the pockets being adapted to receive a thermal medium and hold the thermal medium tightly against a muscle group of the wearer, and each of the pockets further being sized to cover at least a substantial portion of the muscle group. The kit further includes a plurality of thermal media stored within the thermally insulted container.

A thermal therapy method is also provided. The method includes putting on a form-fitting garment includes an elastic material with an outer side, a plurality of pockets attached to the garment on the outer side at locations corresponding to a plurality of muscle groups, each of the pockets being adapted to receive a thermal medium and hold the thermal medium tightly against a muscle group of the wearer, and each of the pockets further being sized to cover at least a substantial portion of the muscle group. Next a thermal medium is inserted into each of the pockets. The garment is worn and the thermal medium is maintained in the pockets until a determined session is complete.

### BRIEF DESCRIPTION OF THE DRAWINGS

The foregoing and other aspects, objects, features and advantages of the invention will become better understood with reference to the following description, appended claims, and accompanying drawings, where:

FIG. 1 illustrates a front plan view of an exemplary short-sleeve shirt having shoulder pockets according to principles of the invention; and

FIG. 2 illustrates a back plan view of the exemplary short-sleeve shirt in FIG. 1 further showing a lumbar pocket according to principles of the invention; and

FIG. 3 illustrates a front plan view of exemplary shorts having quadriceps pockets according to principles of the invention; and

FIG. 4 illustrates a back plan view of the exemplary shorts in FIG. 3 further showing hamstring muscle group pockets according to principles of the invention; and

FIG. 5 illustrates a front plan view of exemplary pants having quadriceps pockets according to principles of the invention; and

FIG. 6 illustrates a back plan view of the exemplary pants in FIG. 3 further showing hamstring and calf muscle group pockets according to principles of the invention; and

FIG. 7 illustrates a front plan view of exemplary pants having stirrups and quadriceps and ankle pockets according to principles of the invention; and

FIG. 8 illustrates an exemplary pocket and thermal media insert for a garment according to principles of the invention; and

FIG. 9 illustrates a rear plan view of exemplary pants having stirrups and hamstring, calf and ankle pockets according to principles of the invention; and

FIG. 10 illustrates a front plan view of an exemplary shirt having elbow and shoulder pockets according to principles of the invention; and

FIG. 11 illustrates a rear plan view of an exemplary shirt having elbow and shoulder pockets according to principles of the invention; and

FIG. 12 illustrates a side plan view of exemplary socks having ankle pockets according to principles of the invention; and

FIG. 13 illustrates a front plan view of an exemplary carrying case for thermal media and garments according to principles of the invention; and

FIG. 14 illustrates a front plan view of an exemplary sleeve having according to principles of the invention.

Those skilled in the art will appreciate that the invention is not limited to the exemplary embodiments depicted in the figures or the shapes, relative sizes, proportions or materials shown in the figures.

### DETAILED DESCRIPTION

The garments described herein may be designed for use by men and/or women, adults and/or children, as an outergarment and/or undergarment. As used herein, pants broadly refer to a garment extending from about the wearer's waist to the legs. Pants may extend to the ankles, calves, knee, below the knee or above the knee. As used herein, shirt broadly refers to a garment that covers all or part of the wearer's upper torso and possibly the wearer's arms and neck. Shirts may include long sleeves, short sleeves, no sleeves, turtle necks, no necks, and may extend below, to or above the wearer's waist. Shorts refer to a garment worn over the pelvic area and the upper part of the upper legs or more, possibly extending as far as mid-calf, but not covering the entire length of the leg.

In a preferred embodiment, garments according to principles of the invention are made close fitting. In a particular preferred embodiment, the garments are comprised of an elastic material and configured to conform to the wearer's body. As used herein, “elastic material” refers to a material that is capable of being easily expanded and resuming former shape. Something that has the ability to resume its former shape after expansion is also referred to herein as being “recoverable.” Preferably, the garments conform to the general area of the body to be supported without excessive stretching that would cause discomfort to the wearer or damage the material. However, the garment may need to be stretched in order to tightly fit and conform to the general area of the body to be treated.

Illustratively, a shape recovery fabric that can be placed in tension and upon release return to its original shape may be utilized. The shape recovery fabric may be comprised of elastic fibers, or a blend of substantially elastic and other fibers. The elastic fibers may comprise spandex or elastane synthetic fibers. Spun from a block copolymer, spandex fibers exploit the high crystallinity and hardness of polyurethane segments, yet remain “rubbery” due to alternating segments of polyethylene glycol. This enables stretching repetitively without breaking and still recovering the original length. The material is also lightweight; abrasion resistant; soft; smooth; supple; resistant to body oils, perspiration, lotions, and detergents. By way of example and not limitation, FIREWALL fabric by Sugoi Performance Products; DuPont Corporation's LYCRA®; and CLEERSPAN® by the Globe Manufacturing Co. are suitable elastic materials that may be used. The elastic fibers may be blended with cotton, polyester, nylon and/or other suitable fibers alone, with other fibers or in various combinations to provide an elastic material suitable for a garment according to principles of the invention.

In a preferred construction, the garment material may be woven to provide equal four-way stretch capabilities, meaning that it stretches equally along all four planar axes. Alternatively, the woven elastic material may be formed so that it stretches more in one direction than another direction, such as allowing the material to stretch more or less in a horizontal direction than in a vertical direction.

As used herein, the term pocket broadly refers to any compartment formed on the garment that is suitable for holding a thermal medium tightly against a body. FIG. 8 provides a close-up plan view of an exemplary pocket 800. The exemplary pocket 800 is comprised of a panel 805 of pocket material attached to the garment along edges, except at least one edge. The unattached edge 810 defines an opening to a compartment between the attached panel 805 and the garment, such as pants, shorts, a shirt or socks. The pocket material may be comprised of the same material as the garment or a different material compatible with the garment. The pocket material may also include moisture impermeable and/or insulating materials and/or layers to protect thermal media within the pocket. Releasable closures, such as hook and loop fasteners, zippers, drawstrings, buttons and/or snaps, may optionally be provided along the unattached edge to seal the pocket in a closed configuration, thereby securing any contents within the compartment.

Pockets are preferably sized and positioned to target determined anatomical areas (muscles, tendons, bones, nerves and other tissue) of the wearer's body. By way of illustration and not limitation, for an adult's garment, a quadriceps pockets 715, 750 may be approximately 8″W×10″L and located on the front part of the middle upper thigh. Major muscle groups targeted by quadriceps pockets 715, 750 include, for example, Sartorius, Rectus Femoris, Vastus Medialis, and Vastus Lateralis. As discussed above, closures may be provided at the open edge of each of the pockets to releasably close the pocket and thereby secure its contents.

The pockets are adapted to hold thermal media 815 for treating targeted areas. Various thermal media now known in the pertinent art and hereafter developed may be utilized for delivering thermal therapy to a targeted area. Such media may include (without limitation) ice and heat packs and pads and hot water bottles. The principles of the invention apply to both cold thermal media and hot thermal media, both of which are intended to come within the scope hereof. Preferably, flexible media is used to enable the media to conform closely to the wearer's physique. In a particular preferred embodiment, Techni Ice™ reusable dry ice packs / gel packs by Techni Ice Australia Pty Ltd are utilized for hot and cold therapies.

Those skilled in the art will appreciate that unlike conventional garments having pockets, garments according to the invention feature a tight form fitting design, with pockets positioned over key muscle groups. In addition, the pockets are sized to receive thermal media and cover all or a substantial portion of the corresponding muscle groups. Furthermore, the tight form fitting design maintains the thermal media firmly against the muscle group being treated to enhance the therapeutic effect. Concomitantly, the garment also provides support and compression to targeted muscle groups, which enhance the recovery process

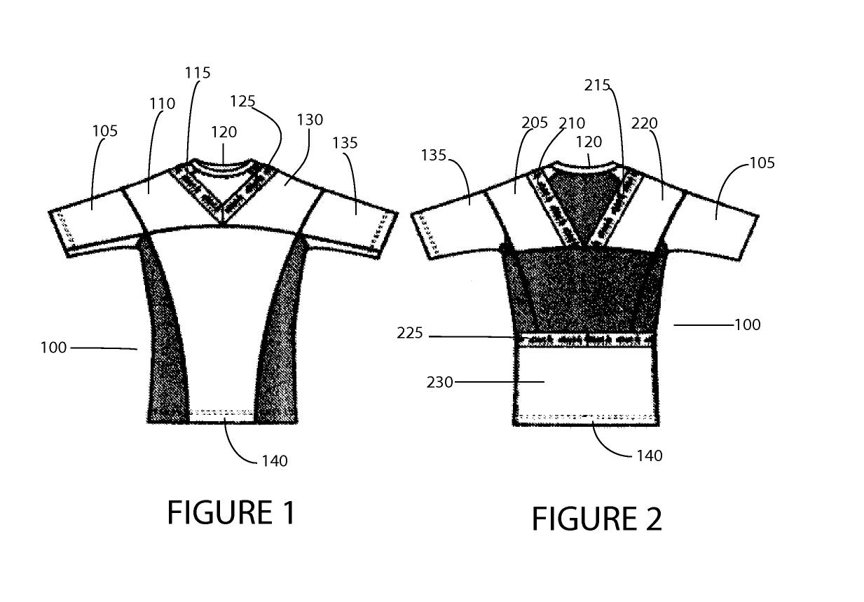

With reference to the drawings, wherein like numerals represent like features, front and rear plan views of an exemplary short-sleeve shirt 100 having shoulder pockets 110, 130 and a lumbar pocket 230 according to principles of the invention are shown in FIGS. 1 and 2, respectively. The shirt 100 may be comprised of an elastic material as described above. An open waist with a waistband 140, a pair of upper arm coverings 105, 135 an arm opening in each arm covering 105, 135 and a neck opening 120 are provided. The shirt 100 is preferably made to fit tightly over and conform to the body. Various panels of material may be stitched together in conventional manner to form the shirt. While the exemplary arm coverings 105, 135 extend to about the wearer's biceps, shorter (or longer) arm coverings may optionally be used. Additionally, optional ankle pockets 725, 740, may be provided along either side, both sides, or the back and/or front of the ankle area of the pants 700.

Each pocket 110, 130 includes an opening with an elastic band 110, 130 to facilitate closure. Other forms of closure, such as hook and loop fasteners, snaps and buttons may be utilized in addition to or in lieu of elastic bands. Additionally, the closure may be omitted without departing from the scope of the invention.

Referring now to FIG. 2, a rear view of the exemplary shirt 100 from FIG. 1 having rear shoulder pockets 205, 220 according to principles of the invention is shown. Additionally, a lumbar pocket 230 is provided. Each pocket 205, 220, 230, includes an opening with an elastic band 210, 215, 225 to facilitate closure. Other forms of closure, such as hook and loop fasteners, snaps and buttons may be utilized in addition to or in lieu of elastic bands. Additionally, the closure may be omitted without departing from the scope of the invention.

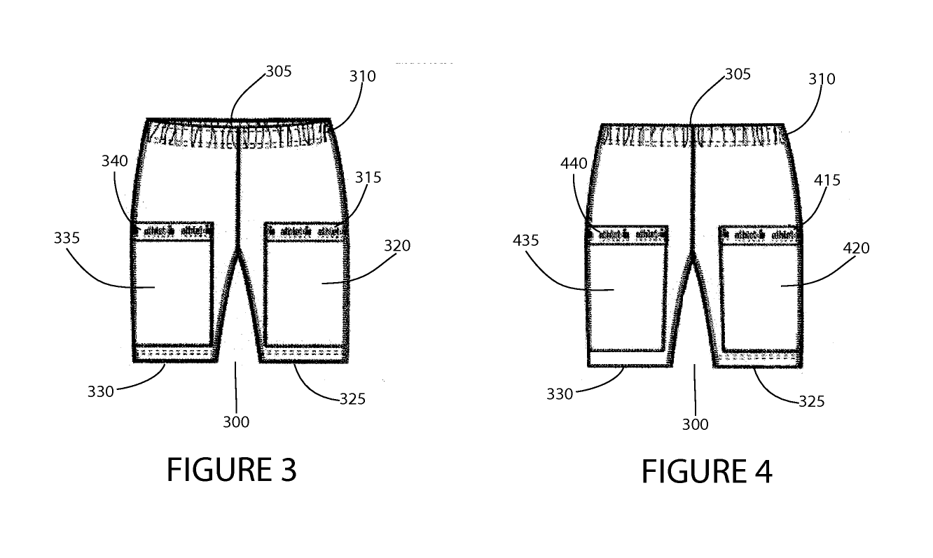

Referring now to FIG. 3, a front view of exemplary stretchable, form-fitting shorts 300 having right and left quadriceps pockets 320, 335 according to principles of the invention is shown. The pants may be comprised of an elastic material as described above. An open waist 305 with a waistband 310, a pair of short leg coverings 325, 330 with a leg opening in each leg covering are provided. The shorts 300 are preferably made to fit tightly over and conform to the body. Various panels of material may be stitched together in conventional manner to form the pants. While the exemplary leg coverings 325, 330 extend to about the wearer's thighs, shorter (or longer) leg coverings may optionally be used.

Each pocket 320, 335 includes an opening with an elastic band 315, 340 to facilitate closure. Other forms of closure, such as hook and loop fasteners, snaps and buttons may be utilized in addition to or in lieu of elastic bands. Additionally, the closure may be omitted without departing from the scope of the invention.

Referring now to FIG. 4, a rear view of the exemplary shorts 300 from FIG. 3 having hamstring pockets 420, 435 according to principles of the invention is shown. Each pocket 420, 435 includes an opening with an elastic band 415, 440 to facilitate closure. Other forms of closure, such as hook and loop fasteners, snaps and buttons may be utilized in addition to or in lieu of elastic bands. Additionally, the closure may be omitted without departing from the scope of the invention.

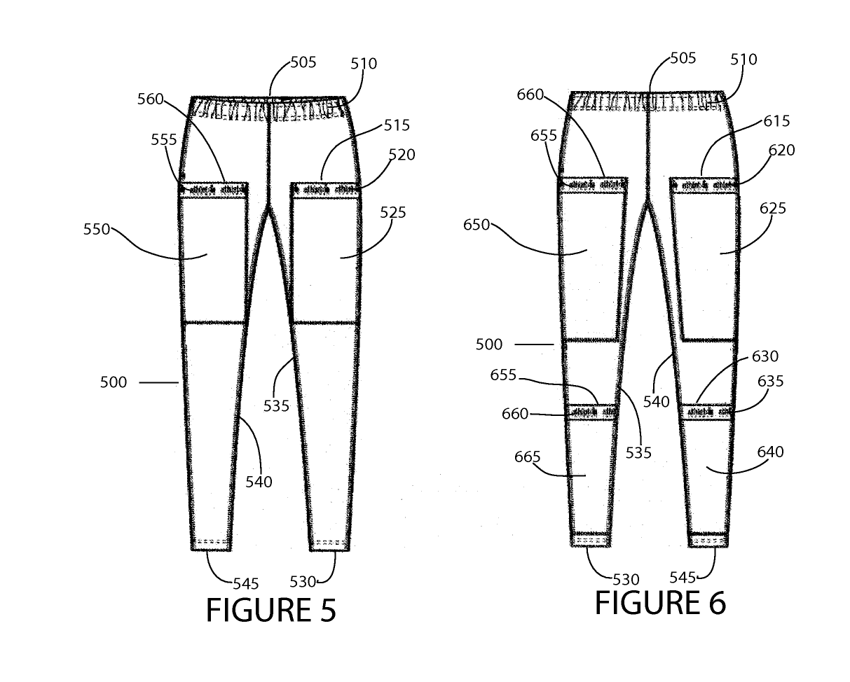

Referring now to FIG. 5, a front view of exemplary stretchable, form-fitting pants 500 having right and left quadriceps pockets 525, 550 according to principles of the invention is shown. The pants may be comprised of an elastic material as described above. An open waist 505 with a waistband 510, a pair of leg coverings 535, 540 and an ankle opening 530, 545 in each leg covering are provided. The pants 500 are preferably made to fit tightly over and conform to the body. Various panels of material may be stitched together in conventional manner to form the pants. While the exemplary leg coverings 535, 540 extend to about the wearer's ankles, shorter (or longer) leg coverings may optionally be used.

Each pocket 525, 550 includes an opening 515, 560 with an elastic band 520, 555 to facilitate closure. Other forms of closure, such as hook and loop fasteners, snaps and buttons may be utilized in addition to or in lieu of elastic bands. Additionally, the closure may be omitted without departing from the scope of the invention.

Referring now to FIG. 6, a rear view of the exemplary pants 500 from FIG. 5 having hamstring pockets 625, 650 according to principles of the invention is shown. Additionally, calf pockets 640, 665 are provided.

Each pocket 625, 640, 650, 665 includes an opening 615, 630, 655, 660 with an elastic band 620, 635, 655, 660 to facilitate closure. Other forms of closure, such as hook and loop fasteners, snaps and buttons may be utilized in addition to or in lieu of elastic bands. Additionally, the closure may be omitted without departing from the scope of the invention.

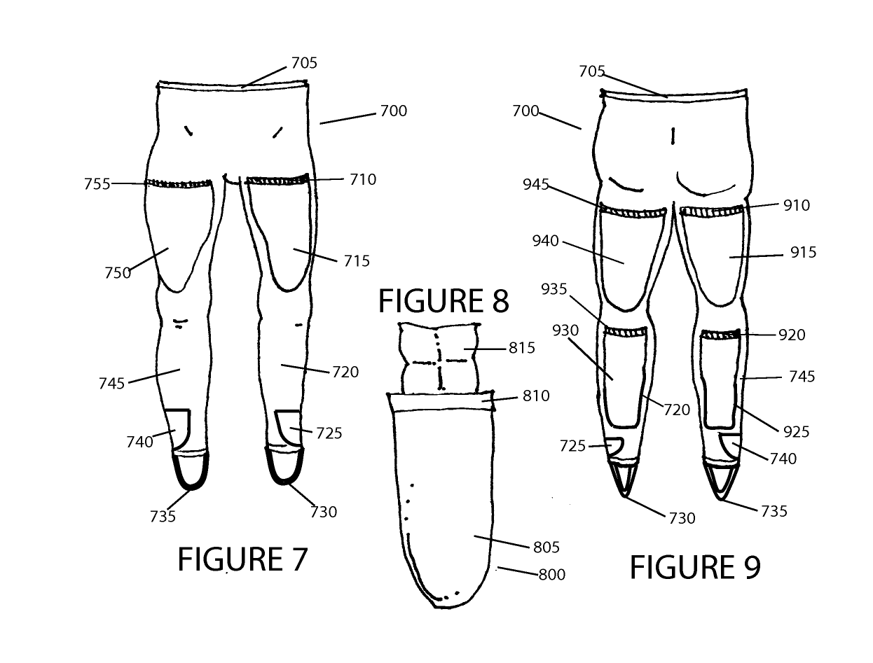

Referring now to FIG. 7, a front view of exemplary stretchable, form-fitting pants 700 having right and left quadriceps pockets 710, 750 according to principles of the invention is shown. The pants may be comprised of an elastic material as described above. An open waist with a waistband 705, a pair of leg coverings 720, 745 and an ankle opening in each leg covering are provided. The pants 700 are preferably made to fit tightly over and conform to the body. Various panels of material may be stitched together in conventional manner to form the pants. While the exemplary leg coverings 720, 745 extend to about the wearer's ankles, shorter (or longer) leg coverings may optionally be used. Optionally, stirrups 730, 735 may be provided to ensure that the bottom of the leg coverings remain in place at or near the ankles when the pants are worn. Additionally, optional ankle pockets 725, 740, may be provided along either side, both sides, or the back and/or front of the ankle area of the pants 700.

Each pocket 715,750 includes an opening with an elastic band 710, 755 to facilitate closure. Other forms of closure, such as hook and loop fasteners, snaps and buttons may be utilized in addition to or in lieu of elastic bands. Additionally, the closure may be omitted without departing from the scope of the invention.

Referring now to FIG. 9, a rear view of the exemplary pants 700 from FIG. 7 having hamstring pockets 915, 940 according to principles of the invention is shown. Additionally, calf pockets 925, 930 and optional ankle pockets 725, 740 are provided.

Each pocket 915, 925, 930, 940, includes an opening with an elastic band 910, 920, 935, 945 to facilitate closure. Other forms of closure, such as hook and loop fasteners, snaps and buttons may be utilized in addition to or in lieu of elastic bands. Additionally, the closure may be omitted without departing from the scope of the invention.

Pockets are preferably sized and positioned to target determined anatomical areas (muscles, tendons, bones, nerves and other tissue) of the wearer's body. By way of illustration and not limitation, for an adult's garment, a quadriceps pockets 715, 750 may be approximately 8″W×10″L and located on the front part of the middle upper thigh. Major muscle groups targeted by quadriceps pockets 715, 750 include, for example, Sartorius, Rectus Femoris, Vastus Medialis, and Vastus Lateralis. As discussed above, closures may be provided at the open edge of each of the pockets to releasably close the pocket and thereby secure its contents.

As another example (but without limitation), hamstring pockets 915, 940 for an adult's garment may be approximately 8″W×10″ L and located on the rear of the middle upper leg. Major muscle groups targeted by hamstring pockets 915, 940 include, for example, Semimembranosis and Biceps Femoris. As discussed above, closures may be provided at the open edge of each of the pockets to releasably close the pocket and thereby secure its contents.

As yet another example (but without limitation), calf muscle pockets 925, 930 for an adult's garment may be approximately 8″L×6″W and located on the rear of the lower leg. Major muscle groups and (optionally) tendons targeted by calf muscle pockets 925, 930 include, for example, Gastrocnemius, Soleus and Achilles Tendons. If the Achilles Tendon is targeted, the calf muscle pockets 925, 930 may extend to the ankles. As discussed above, closures may be provided at the open edge of each of the pockets to releasably close the pocket and thereby secure its contents.

The pockets identified above are examples, but not an exhaustive list, of pockets that may be included on pants in accordance with the principles of the invention. Such pants may include one or more of any of the aforementioned pockets, and/or pockets positioned to target other anatomical regions.

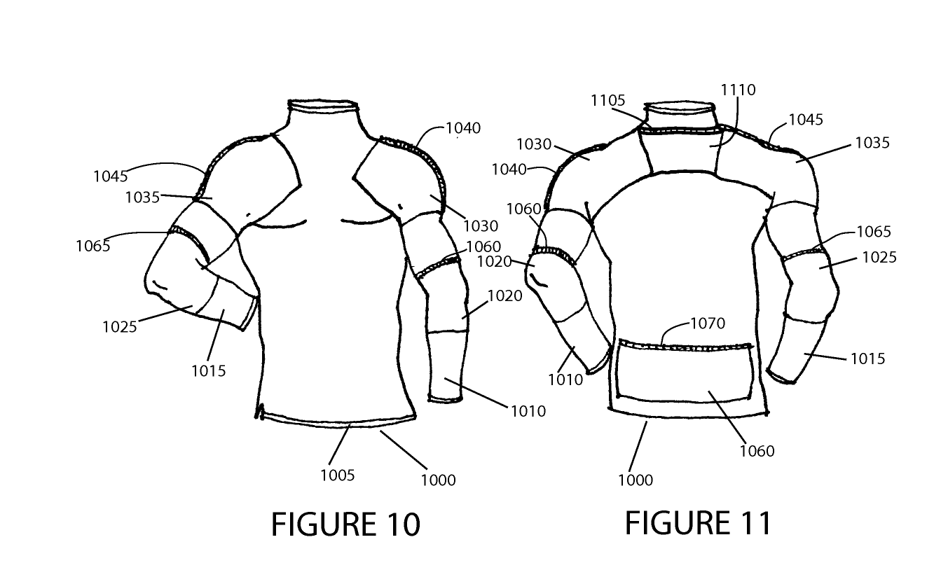

Referring now to FIG. 10, a front view of an exemplary form-fitting, stretchable, pull-over shirt 1000 having elbow 1020, 1025 and shoulder 1030, 1035 pockets according to principles of the invention is shown. The shirt 1000 may be comprised of an elastic material as described above. An open waist with a waistband 1005, an open neck, a pair of sleeves 1010, 1015 and a wrist opening in each arm covering are provided. The shirt 1000 is preferably made to fit tightly over and conform to the body. Various panels of material may be stitched together in conventional manner to form the shirt. While the exemplary sleeves 1010, 1015 extend to about the wearer's wrists, shorter (or longer) sleeves may optionally be used.

Each of the elbow 1020, 1025 and shoulder 1030, 1035 pockets includes an opening with an elastic band 1040, 1060, 1045, 1065 to facilitate closure. Other forms of closure, such as hook and loop fasteners, snaps and buttons may be utilized in addition to or in lieu of elastic bands. Additionally, the closure may be omitted without departing from the scope of the invention.

Now referring now to FIG. 11, a rear view of the exemplary shirt 1000 having elbow 1020, 1025 and shoulder 1030, 1035 pockets, as well as a lower back (i.e., lumbar) pocket 1060, and a trapezius 1110 pocket, according to principles of the invention is shown. The lower back pocket 1060 may optionally include an opening with an elastic band 1070 to facilitate closure. Likewise, the trapezius pocket 1110 may optionally include an opening with an elastic band 1105 to facilitate closure. Other forms of closure, such as hook and loop fasteners, snaps and buttons may be utilized in addition to or in lieu of elastic bands. Additionally, the closures may be omitted without departing from the scope of the invention.

The shirt pockets are preferably sized and positioned to target determined anatomical areas (muscles, tendons, bones, nerves and other tissue) of the wearer's body. By way of illustration and not limitation, for an adult's garment, shoulder pockets 1030, 1035 may be approximately 6″W×8″L and located on the topside of the upper sleeve. Major muscle groups targeted by shoulder pockets 1030, 1035 may include Trapezius, Deltoid, Biceps brachii, Brachialis, Brachial plexus, Brachioradialis, Coracobrachialis, Latissimus dorsi, Subclavius, Subscapularis, Supraspinatus, Teres major, Triceps brachii.

As with the pants pockets, the shirt pockets are adapted to hold thermal media 815 for treating targeted areas. Various thermal media now known in the pertinent art and hereafter developed may be utilized for delivering thermal therapy to a targeted area. Such media may include (without limitation) ice and heat packs and pads and hot water bottles. The principles of the invention apply to both cold thermal media and hot thermal media, both of which are intended to come within the scope hereof. Preferably, flexible media is used to enable the media to conform closely to the wearer's physique. In a particular preferred embodiment, Techni Ice reusable dry ice packs / gel packs by Techni Ice Australia Pty Ltd are utilized for hot and cold therapies.

As another example (but without limitation), elbow pockets 1020, 1025 for an adult's garment may be approximately 6″W×6″ L and located on the outer elbow region of each sleeve. Major muscle groups targeted by elbow pockets 1020, 1025 may include, for example, portions of Anconeus (cubitalis rolani), Brachioradialis, Extensor carpi radialis brevis, Extensor carpi radialis longus, Extensor carpi ulnaris, Extensor digiti minimi, Extensor digitorum, Flexor carpi ulnaris, Biceps brachii, Brachialis, Brachioradialis, Flexor carpi radialis, Flexor carpi ulnaris, Palmaris longus, and Pronator teres.

As yet another example (but without limitation), a lower back (e.g., lumbar) pocket 1060 for an adult's garment may be approximately 12″L×6″W and located on the lower portion of the rear of the shirt 1000. Major muscle groups targeted by the lower back pocket 1060 may include, for example, portions of Quadratus Lumborum, Erector Spinae, Latissimus Dorsi, Trapezius, and External Oblique.

The pockets identified above are examples, but not an exhaustive list, of pockets that may be included on shirts in accordance with the principles of the invention. Such pants may include one or more of any of the aforementioned pockets, and/or pockets positioned to target other anatomical regions, such as the chest (pectoral), front waist (abdominal) and other areas. Additionally, in an embodiment with an extended neck (i.e., a turtleneck), a pocket may be provided in the front and/or rear of the neck region.

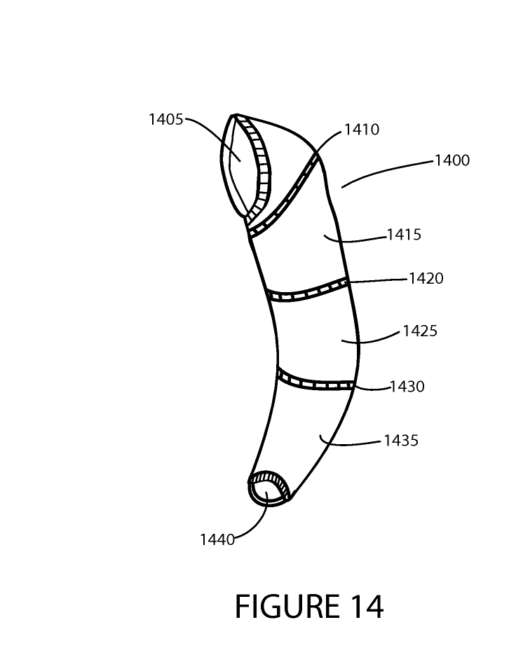

Referring now to FIG. 14, a front plan view of an exemplary form-fitting, stretchable, pull-on sleeve 1400 having an upper arm pocket 1415, elbow pocket 1425, and forearm pocket 1435. The pockets are sized and oriented to receive thermal media and maintain the media tightly against targeted muscle groups, such as biceps brachii and triceps brachii in the upper arm, the elbow joint, and forearm muscles including the flexors and extensors of the digits, a flexor of the elbow (brachioradialis), and pronators and supinators that turn the hand to face down or upwards, respectively. The sleeve 1400 may be comprised of an elastic material as described above. An open wrist with a wristband 1440 and an open shoulder 1405 with a shoulder band are provided. The sleeve 1400 is preferably made to fit tightly over and conform to the user's arm. Various panels of material may be stitched together in conventional manner to form the sleeve 1400. While the exemplary sleeve 1400 shown in FIG. 14 extends from the wearer's shoulder to about the wearer's wrists, shorter (or longer) sleeves may optionally be used within the spirit and scope of the invention.

Each of the upper arm pocket 1415, elbow pocket 1425, and forearm pocket 1435 includes an opening with an elastic band 1410, 1420, 1430 to facilitate closure. Other forms of closure, such as hook and loop fasteners, snaps and buttons may be utilized in addition to or in lieu of elastic bands. Additionally, the closure may be omitted without departing from the scope of the invention.

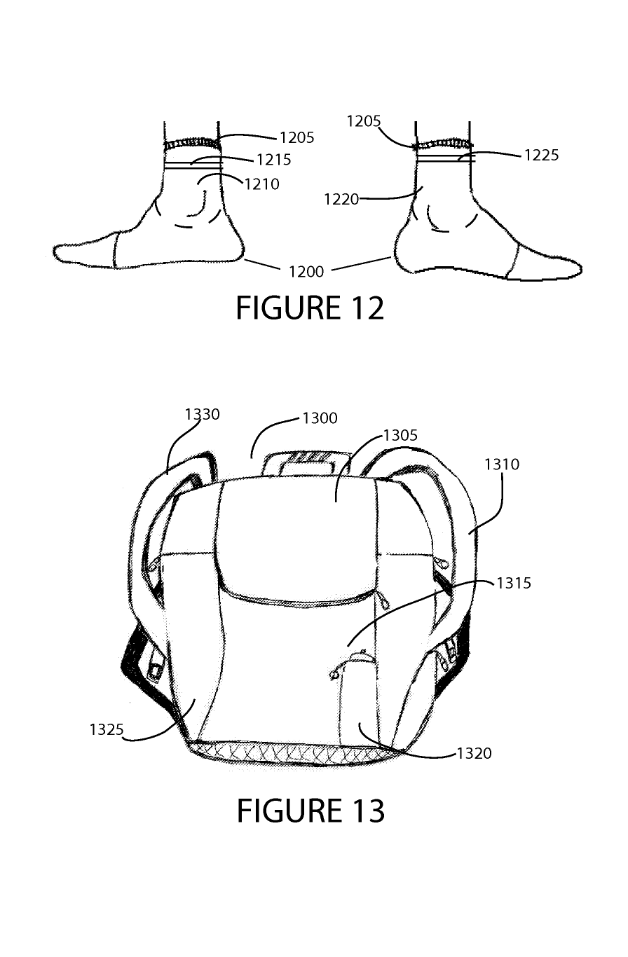

In yet another embodiment, a garment according to principles of the invention may comprise hosiery such as a sock 1200, as shown in FIG. 12, with one or more pockets 1210, 1220 in the area of the Achilles tendon. An opening with an elastic band 1205 is provided in a conventional manner. Each pocket 1210, 1220 may optionally include an opening with an elastic band 1215, 1225 to facilitate closure. Other forms of closure, such as hook and loop fasteners, snaps and buttons may be utilized in addition to or in lieu of elastic bands. Additionally, the closure may be omitted without departing from the scope of the invention.

Yet another embodiment comprises one or more garments as described above, in accordance with principles of the invention, in a thermal kit. The kit includes a thermal container such as a duffel bag or backpack with a thermally insulated compartment for storing chilled/frozen thermal media (or heated thermal media) for an extended period of time, and another compartment (or the same compartment) for storing the garment. As shown in FIG. 13, an exemplary backpack 1300 is comprised of a container with a releasable closure 1305, and attached shoulder straps 1310, 1330. The closure 1305 provides access to one or more internal compartments, such as an insulated compartment for storing thermal media 1315 and mesh compartments 1325 for storing other items. Optionally, the backpack 1300 may also include a water bottle. The most common materials for such packs are canvas and nylon, either ripstop, ballistic or sack cloth fabrics. Thus, an athlete may bring the kit with him or her to a training session or an event. The heated or chilled thermal media will maintain an effective hot or cold temperature for several hours within the insulated compartment 1315 of the container. After training or completing the event, the athlete may immediately commence thermal therapy using the garment and thermal media, targeting specific muscle groups without the inconvenience and discomfort of an ice bath.

The invention provides several advantages. First, the invention is portable and facilitates thermal therapy immediately after strenuous activity, which enhances the restoration process. Another advantage is that fatigued and strained muscle groups and other anatomical areas may be targeted with thermal therapy by inserting thermal media into corresponding pockets, while other part of the body may remain at a comfortable ambient temperature. Yet another advantage is that the garment provides support and compression to targeted muscle groups, which enhance the recovery process.

While an exemplary embodiment of the invention has been described, it should be apparent that modifications and variations thereto are possible, all of which fall within the true spirit and scope of the invention. With respect to the above description then, it is to be realized that the optimum relationships for the components of the invention and steps of the process, including variations in form, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A form-fitting garment comprising an elastic material with an outer side, a plurality of pockets attached to said garment on said outer side at locations corresponding to a plurality of muscle groups, each of said pockets being adapted to receive a thermal medium and hold the thermal medium tightly against a muscle group of the wearer, and each of said pockets further being sized to cover at least a substantial portion of the muscle group.

2. A form-fitting garment according to claim 1, wherein said garment comprises a short-sleeve shirt and said plurality of pockets include shoulder pockets, said shoulder pockets being positioned on areas of the shirt corresponding to shoulders of an intended wearer.

3. A form-fitting garment according to claim 1, wherein said garment comprises a short-sleeve shirt and said plurality of pockets include a lumbar pocket, said lumbar pocket being positioned on an area of the shirt corresponding to a lumbar region of an intended wearer.

4. A form-fitting garment according to claim 1, wherein said garment comprises a short-sleeve shirt and said plurality of pockets include a trapezius pocket, said trapezius pocket being positioned on an area of the shirt corresponding to a trapezius region of an intended wearer.

5. A form-fitting garment according to claim 1, wherein said garment comprises a long-sleeve shirt and said plurality of pockets include shoulder pockets, said shoulder pockets being positioned on areas of the shirt corresponding to shoulders of an intended wearer.

6. A form-fitting garment according to claim 1, wherein said garment comprises a long-sleeve shirt and said plurality of pockets include a lumbar pocket, said lumbar pocket being positioned on an area of the shirt corresponding to a lumbar region of an intended wearer.

7. A form-fitting garment according to claim 1, wherein said garment comprises a long-sleeve shirt and said plurality of pockets include a trapezius pocket, said lumbar pocket being positioned on an area of the shirt corresponding to a trapezius region of an intended wearer.

8. A form-fitting garment according to claim 1, wherein said garment comprises a long-sleeve shirt and said plurality of pockets include upper arm pockets, said upper arm pockets being positioned on an area of the shirt corresponding to upper arm regions of an intended wearer.

9. A form-fitting garment according to claim 1, wherein said garment comprises a long-sleeve shirt and said plurality of pockets include elbow pockets, said elbow pockets being positioned on an area of the shirt corresponding to elbow regions of an intended wearer.

10. A form-fitting garment according to claim 1, wherein said garment comprises a long-sleeve shirt and said plurality of pockets include forearm pockets, said forearm pockets being positioned on an area of the shirt corresponding to forearm regions of an intended wearer.

11. A form-fitting garment according to claim 1, wherein said garment comprises shorts and said plurality of pockets include quadriceps pockets, said quadriceps pocket being positioned on an area of the shorts corresponding to quadriceps regions of an intended wearer.

12. A form-fitting garment according to claim 1, wherein said garment comprises shorts and said plurality of pockets include hamstring pockets, said hamstring pocket being positioned on an area of the shorts corresponding to hamstring regions of an intended wearer.

13. A form-fitting garment according to claim 1, wherein said garment comprises pants and said plurality of pockets include quadriceps pockets, said quadriceps pocket being positioned on an area of the pants corresponding to quadriceps regions of an intended wearer.

14. A form-fitting garment according to claim 1, wherein said garment comprises pants and said plurality of pockets include hamstring pockets, said hamstring pocket being positioned on an area of the pants corresponding to hamstring regions of an intended wearer.

15. A form-fitting garment according to claim 1, wherein said garment comprises pants and said plurality of pockets include calf pockets, said calf pocket being positioned on an area of the pants corresponding to calf regions of an intended wearer.

16. A form-fitting garment according to claim 1, wherein said garment comprises pants and said plurality of pockets include ankle pockets, said ankle pocket being positioned on an area of the pants corresponding to ankle regions of an intended wearer.

17. A form-fitting garment according to claim 1, wherein said garment comprises a pair of socks and said plurality of pockets include ankle pockets, said ankle pockets being positioned on areas of the socks corresponding to ankle regions of an intended wearer.

18. A form-fitting garment according to claim 1, wherein said garment comprises a sleeve and said plurality of pockets include an upper arm pocket and an elbow pocket said upper arm pocket being positioned on areas of the sleeve corresponding to upper arm regions of an intended wearer, and said elbow pocket being positioned on areas of the sleeve corresponding to elbow regions of an intended wearer.

19. A thermal therapy kit comprising
a thermally insulated container adapted for storing a thermal medium and a form fitting garment;
a form-fitting garment stored in said thermally insulated container and comprising an elastic material with an outer side, a plurality of pockets attached to said garment on said outer side at locations corresponding to a plurality of muscle groups, each of said pockets being adapted to receive a thermal medium and hold the thermal medium tightly against a muscle group of the wearer, and each of said pockets further being sized to cover at least a substantial portion of the muscle group; and
a plurality of thermal media stored within said thermally insulted container.

20. A thermal therapy method comprising:
putting on a form-fitting garment comprising an elastic material with an outer side, a plurality of pockets attached to said garment on said outer side at locations corresponding to a plurality of muscle groups, each of said pockets being adapted to receive a thermal medium and hold the thermal medium tightly against a muscle group of the wearer, and each of said pockets further being sized to cover at least a substantial portion of the muscle group;
inserting a thermal medium into each of said pockets;
wearing said garment and maintaining said thermal medium in each of said pockets until a determined session is complete.
